(19) United States
(12) Patent Application Publication
(10) Pub. No.: US 2024/0158752 A1
(43) Pub. Date: May 16, 2024

(54) MEDIUM AND METHOD FOR PRODUCING A BONE MARROW RECONSTITUTION

(71) Applicants: ETABLISSEMENT FRANÇAIS DU SANG, LA PLAINE SAINT DENIS (FR); INSTITUT NATIONAL DE LA SANTE ET DE LA RECHERCHE MEDICALE (INSERM), PARIS (FR); UNIVERSITE TOULOUSE III-PAUL SABATIER, TOULOUSE CEDEX 9 (FR); ECOLE NATIONALE VÉTÉRINAIRE DE TOULOUSE (ENVT), TOULOUSE (FR)

(72) Inventors: Mélanie Alexandra GADELORGE, LAGARDELLE SUR LÈZE (FR); Nicolas Lucien ESPAGNOLLE, LAPEYROUSE FOSSAT (FR); Frédéric DESCHASEAUX, NERAC (FR)

(21) Appl. No.: 18/550,889

(22) PCT Filed: Mar. 18, 2022
(86) PCT No.: PCT/EP2022/057229

§ 371 Date: Sep. 15, 2023

(30) Foreign Application Priority Data
Mar. 18, 2021 (FR) 2102708

(51) Int. Cl. C12N 5/077; A61P 19/00; C12N 5/071; C12N 5/0775

(52) U.S. Cl. CPC C12N 5/0654; A61P 19/00; C12N 5/0653; C12N 5/0663; C12N 5/0692; C12N 5/0697; C12N 2500/25; C12N 2500/36; C12N 2500/38; C12N 2501/155; C12N 2501/165; C12N 2501/33; C12N 2513/00

(57) ABSTRACT

Disclosed is a culture medium and a culture method for obtaining, in a single step and in the same culture container, cells differentiated into osteoblasts and adipocytes, as well as a network of organized endothelial cells from the same pool of mesenchymal stem cells and/or a mixture of mesenchymal stem cells, endothelial progenitor cells and endothelial cells selected beforehand and amplified simultaneously in the same culture container from a single sample. Also disclosed is a composition obtained by the method, to a bone marrow reconstitution and to uses thereof.

The present invention relates to a culture medium and to a culture method for obtaining cells that are differentiated into osteoblasts and adipocytes in a single step and in one same culture container, together with a network of organized endothelial cells from one same pool of mesenchymal stem cells and/or a mixture of mesenchymal stem cells, endothelial progenitor cells and endothelial cells, previously selected and amplified simultaneously in one same culture container from a single sample. It also relates to a composition obtained with said method, to a bone marrow reconstitution and to uses thereof.

The present invention also relates to a culture medium and a culture method allowing the culture and/or differentiation into osteoblasts and adipocytes of mesenchymal stem cells, and also allowing the organization of a network of endothelial cells. It further relates to a composition obtained with said method, to a bone marrow reconstitution and to uses thereof.

The reproduction of human adult bone marrow ex vivo is increasingly more described in the literature to overcome the use of animal models that are costly, time consuming and dependent on the species barrier. Recent studies have started evidencing 3D models of co-cultures grouping together osteoblastic and endothelial compartments, generally of cells derived from cell lines. For example, the endothelial compartment playing an active role in the proliferation of hematopoietic stem cells (HSCs) is often incorporated via endothelial cell lines of HUVEC type. Other models require a step in mice, to allow vascularization or functional approaches. In addition, most of the time these co-cultures are obtained in several steps (osteoblastic differentiation of mesenchymal stem cells at a first stage, followed by assembling with a second pool of endothelial cells at a second stage). Also, bone marrow adipose tissue is often ignored in these studies despite the increasing functional importance thereof described in the literature.

The inventors have created a novel model exhaustively incorporating all cellular and non-hematopoietic microenvironmental parameters of human bone marrow. It comprises bone marrow adipose tissue, the osteoblastic compartment and vascular compartment also called endothelial compartment. The culture medium developed by the inventors allows the simultaneous obtaining of these 3 non-hematopoietic compartments of human bone marrow and in particular from a single sample, in a single step, in the same culture container without the use of a cell line. This makes it possible to generate ex vivo human bone marrow comprising the osteoblastic, adipocyte and endothelial compartments in 2D, but also in 3D in particular via the use of a biomaterial. In addition, via self-organization, this ex vivo bone marrow is able to form a 3D structure of spheroid/organoid type.

Additionally, in preceding studies, the differentiation of mesenchymal stem cells into adipocytes and osteoblasts necessitated the separate performing of the two differentiations in two different media. Not only does the medium developed by the inventors allow the performing of these two differentiation routes at the same time, but it also promotes the organization of an endothelial network in the same medium, and in the same culture well, without posterior assembling of cells, thereby largely simplifying handling operations required in particular for obtaining a bone marrow reconstitution.

The invention therefore concerns a culture medium allowing the obtaining of cells in a single step and in one same culture container, that are differentiated into osteoblasts and adipocytes, as well as a network of organized endothelial cells, from one same pool of mesenchymal stem cells and/or a mixture of mesenchymal stem cells, endothelial progenitor cells and endothelial cells, comprising:
a) fetal calf serum (FCS), and/or platelet lysate (PL);
b) 10-100 μM ascorbic acid;
c) 10-100 ng/mL «Bone Morphogenetic Protein 7» (BMP7);
d) 1-10 μg/mL insulin;
e) 1-50 μg/mL apotransferrin;
f) 1-100 ng/mL vascular endothelial growth factor (VEGF), and optionally
g) 0.01-0.5% (v/v) intralipids.

The invention therefore concerns a culture medium for the culture and/or differentiation of mesenchymal stem cells into osteoblasts and adipocytes, and optionally allowing the organization of a network of endothelial cells, comprising:
a) fetal calf serum (FCS) and/or platelet lysate (PL);
b) 10-100 μM ascorbic acid;
c) 10-100 ng/mL «Bone Morphogenetic Protein 7» (BMP7),
d) 1-10 μg/mL insulin,
e) 1-50 μg/mL apotransferrin,
f) 1-100 ng/mL vascular endothelial growth factor (VEGF).

In a second aspect, the invention concerns an in vitro culture method of mesenchymal stem cells, comprising the steps of:
a) seeding the mesenchymal stem cells in a culture medium such as defined above; and
b) culturing said mesenchymal stem cells.

The invention concerns an in vitro culture method of mesenchymal stem cells and/or a mixture of mesenchymal stem cells, endothelial progenitor cells and endothelial cells, comprising the steps of:
a) seeding the mesenchymal stem cells and/or the mixture of mesenchymal stem cells, endothelial progenitor cells and endothelial cells in a culture medium such as defined above; and
b) culturing said mesenchymal stem cells and/or said mixture of mesenchymal stem cells, endothelial progenitor cells and endothelial cells.

Preferably, with this method it is possible, in a single step, in one same culture container, to obtain cells differentiated into osteoblasts and adipocytes, as well as a network of organized endothelial cells from one same pool of mesenchymal stem cells and/or a mixture of mesenchymal stem cells, endothelial progenitor cells and endothelial cells.

The invention concerns a method for producing a bone marrow reconstitution, comprising the in vitro culture method of mesenchymal stem cells according to the invention.

The invention concerns a method for producing a bone marrow reconstitution, comprising the in vitro culture method of mesenchymal stem cells and/or a mixture of mesenchymal stem cells, endothelial progenitor cells and endothelial cells according to the invention.

A further subject of the invention is a bone marrow reconstitution obtained with the method of the invention. The invention also concerns a bone marrow reconstitution comprising osteoblasts, adipocytes, and vessel-forming endothelial cells. The invention further concerns a bone marrow reconstitution comprising osteoblasts, adipocytes, and a network of endothelial cells.

A further subject of the invention is a composition, preferably for injection, comprising cells obtained with the method of the invention.

The invention also concerns a bone marrow reconstitution according to the invention or a composition of the invention for use thereof in the treatment of diseases, in particular diseases affecting the integrity of the bone marrow and/or related to a hematopoiesis disorder.

Finally, the invention concerns the use of a bone marrow reconstitution of the invention as model for physiology, physiopathology studies, for the testing of compounds and/or the testing of physical and mechanical conditions.

The invention also pertains to the use of a bone marrow reconstitution of the invention in a prosthesis or medical device.

### DETAILED DESCRIPTION OF THE INVENTION

Culture Medium

By «medium for the culture and/or differentiation into osteoblasts and adipocytes of mesenchymal stem cells», it is meant a medium adapted for the culture and/or differentiation into osteoblasts and adipocytes of mesenchymal stem cells. Preferably, this medium is a medium for the culture and/or differentiation into osteoblasts and adipocytes of mesenchymal stem cells and for the formation of a vascular network by endothelial progenitor cells and/or endothelial cells.

By «medium for the culture and differentiation into osteoblasts and adipocytes of mesenchymal stem cells», it is meant a medium adapted for the culture and simultaneous differentiation into osteoblasts and adipocytes of one same pool of mesenchymal stem cells, in a single step, in one same culture container, without assembling of cells. Preferably, this medium is a medium for the culture and differentiation into osteoblasts and adipocytes of mesenchymal stem cells allowing the organization of a network of endothelial cells.

Preferably, this medium allows the simultaneous differentiation of mesenchymal stem cells into osteoblasts and adipocytes. Preferably, this medium allows the simultaneous differentiation of mesenchymal stem cells into osteoblasts and adipocytes and also the organization of an endothelial network, in a single step, in a single culture container, without posterior assembling of cells. It can be in different forms but it is preferably liquid and allows the culture of eukaryote cells, in particular of mammalian cells and more particularly of human cells.

Such as provided herein, the term «culture» relates to the multiplication of cultured cells.

The term «differentiation» concerns the acquisition by cells, cultured in a culture medium, of cell characteristics which are not contained in the cells initially used to seed the culture medium. As provided herein, «differentiation» particularly designates the acquisition of characteristics particularly placing the cells on the adipocyte or osteoblastic pathway.

By «mesenchymal stem cells» also called «mesenchymal stromal cells», or MSCs, it is meant stromal cells of mesodermal origin. They are phenotype characterized by the co-expression of a certain number of markers such as CD73, CD90, CD105, CD146 for example, and lack of expression of other markers in particular CD45 and CD34. They can be derived from the bone marrow, adipose tissue, or umbilical cord blood of mammals. Mesenchymal stem cells can be taken from rodents or primates, and in particular are of murine or human origin.

In one preferred embodiment, the mesenchymal stem cells are derived from primary cultures. By «primary culture» it is meant a culture of cells directly derived from the tissue and/or cells of an individual.

By «endothelial progenitor cells» it is meant cells involved in endothelial differentiation but which are not yet recognizable as endothelial cells under a microscope. They are phenotype characterized by the expression of a certain number of markers such as CD133, CD34, CD31, VEGFR2.

By «endothelial cells», it is meant cells that are fully differentiated on the endothelial pathway and therefore recognizable as endothelial cells under a microscope. They are phenotype characterized by the expression of a certain number of markers such as CD31, VE-Cadherine, von Willebrand factor, VEGFR2.

Endothelial progenitor cells and endothelial cells have the capability of organizing themselves into a network of endothelial cells, or vascular network, and therefore of organizing themselves into vessels.

The endothelial progenitor cells and endothelial cells of the invention can be obtained for example from bone marrow mononuclear cells.

By «individual» it is meant a subject of an animal species, in particular of mammals. In one embodiment of the invention, the individual is a primate or a rodent, preferably a mouse or a human being.

By «osteoblasts» it is meant cells expressing the markers Runx2, DSX, ESP, BSP, DLX5 and/or Osterix (OSX). The osteoblastic phenotype can be evaluated under phase-contrast microscopy by assessing extent of mineralization with alizarin-red red staining, by immunohistochemical evidencing of alkaline phosphatase activity (ALP) with the chemical reaction to Naphtol AS-BI phosphate, by immunofluorescence with evidencing of osteocalcin, osteopontin, PAL and OSX.

By «adipocytes» it is meant cells expressing the markers LPL, PPARγ, AdipoQ and/or cells able to be detected with a BODIPY fluorescent probe marking the lipids contained in lipid vacuoles. The adipocyte nature of cells can be verified by phase-contrast microscopy analysis showing the presence of lipid vacuoles, and by immunohistochemical staining with Oil Red O marking the lipids contained in lipid vacuoles.

The culture medium of the invention is composed of a base medium supplemented with various compositions and/or compounds.

Preferably, the base medium allows the culture of eukaryote cells such as mammalian cells and in particular human cells. Said culture media are well known to persons skilled in the art. Preferably the base medium is selected from among De DMEM, MEM-α, Ham's F-12, RPMI 1640, IMDM and combinations thereof. Preferably, the base medium is MEM-α.

In one embodiment, the base medium of the invention is supplemented with fetal calf serum (FCS), preferably 0.5 to 5% (v/v) FCS, and more particularly 2% (v/v) FCS. This medium may also comprise intralipids, preferably from 0.01% to 1% (v/v) intralipids, preferably from 0.01 to 0.5% (v/v) intralipids, and more particularly 0.04% (v/v) intralipids.

In a second embodiment, the base medium of the invention is supplemented with platelet lysate (PL), preferably from 0.5 to 5% (v/v) PL, and more particularly 1% (v/v) PL.

In a third embodiment, the base medium of the invention is supplemented with fetal calf serum (FCS) and platelet lysate (PL). This medium may also comprise intralipids, preferably from 0.01% to 1% (v/v) intralipids, preferably from 0.01 to 0.5% (v/v) intralipids, and more particularly 0.04% (v/v) intralipids.

The fetal calf serum and platelet lysate are preferably sterile before being used in the culture medium.

The base medium of the invention is also supplemented with 10 to 100 μM ascorbic acid, 10 to 100 ng/mL «Bone Morphogenetic Protein 7 » (BMP7), 1 to 10 μg/mL insulin, 1 to 50 μg/mL apotransferrin, and 1 to 100 ng/mL vascular endothelial growth factor (VEGF).

Preferably, the medium of the invention is supplemented with 30 to 70 μM ascorbic acid, more preferably with 45 to 55 μM ascorbic acid.

Preferably, the medium of the invention is supplemented with 30 to 70 ng/mL BMP7, more preferably with 45 to 55 ng/mL BMP7. Preferably, the BMP7 of the invention is a recombinant human protein.

Preferably, the medium of the invention is supplemented with 3 to 7 μg/mL insulin, more preferably with 4.5 to 5.5 μg/mL insulin. Preferably the insulin of the invention is recombinant human insulin.

Preferably, the medium of the invention is supplemented with 8 to 12 μg/mL apotransferrin, more preferably with 9 to 11 μg/mL apotransferrin. Preferably, the apotransferrin of the invention is recombinant human apotransferrin.

Preferably, the medium of the invention is supplemented with 1 to 20 ng/mL vascular endothelial growth factor (VEGF), more preferably with 5 to 15 ng/mL VEGF. Preferably, the VEGF of the invention is recombinant human VEGF.

The proteins used in the medium are preferably of recombinant origin and used in purified form.

The culture medium of the invention can be sterile or filtered before use. The culture medium of the invention can be used in varied culture methods.

In Vitro Culture Method

The invention concerns an in vitro culture method of mesenchymal stem cells, comprising the steps of:
a) seeding the mesenchymal stem cells in a culture medium such as defined above; and
b) culturing said mesenchymal stem cells.

The invention also concerns an in vitro culture method of mesenchymal stem cells and/or a mixture of mesenchymal stem cells, endothelial progenitor cells and endothelial cells, comprising the steps of:
a) seeding the mesenchymal stem cells and/or the mixture of mesenchymal stem cells, endothelial progenitor cells and endothelial cells in a culture medium such as defined above; and
b) culturing said mesenchymal stem cells and/or said mixture of mesenchymal stem cells, endothelial progenitor cells and endothelial cells.

Preferably, these methods allow the obtaining, in a single step and in one same culture container, of cells differentiated into osteoblasts and adipocytes as well as a network of organized endothelial cells from one same pool of mesenchymal stem cells and/or a mixture of mesenchymal stem cells, endothelial progenitor cells and endothelial cells.

In these methods of the invention, endothelial progenitor cells and endothelial cells can also be cultured in the culture medium, at the same time as said mesenchymal stem cells.

In one embodiment, the culture is adherent monolayer culture, non-adherent or in suspension or in the presence of a biomaterial (in solid form or in the form of a gel). In another embodiment of the methods of the invention, the cells are cultured in suspension.

The culture can be conducted continuously or discontinuously, in batch, in fed-batch or perfusion bioreactor, or on a microfluidic chip.

In the in vitro culture method of the invention, one portion of the mesenchymal stem cells is differentiated into osteoblasts and another portion of the mesenchymal stem cells is differentiated into adipocytes, preferably simultaneously, in a single step, in the same medium and in the same culture container, without posterior assembling of cells, and preferably from a single sample.

Preferably, in the in vitro culture methods of the invention, the endothelial progenitor cells and endothelial cells organize themselves into vessels in the culture medium, at the same time as said mesenchymal stem cells differentiate.

Preferably, all the cells in the method of the invention are derived from one same animal species, in particular from a mammal. In one embodiment of the invention, the cells are cells of rodents or primates, preferably human cells.

Endothelial progenitor cells and/or endothelial cells can be cultured in the culture medium at the same time as said mesenchymal stem cells.

Endothelial progenitor cells and/or endothelial cells contained in the same initial pool of cells as the mesenchymal stem cells and derived from the same donor can be cultured in the culture medium at the same time as said mesenchymal stem cells.

In one preferred embodiment, the cells in the culture methods of the invention are primary cells.

In one preferred embodiment, the mesenchymal stem cells are derived from primary cultures.

In one preferred embodiment, the endothelial progenitor cells and the endothelial cells are derived from primary cultures.

Preferably, the mesenchymal stem cells, the endothelial progenitor cells and the endothelial cells are derived from primary cultures, preferably derived from the same individual.

In one embodiment, the mesenchymal stem cells, the endothelial progenitor cells, and the endothelial cells are derived from a single sample from an individual. Preferably, the mesenchymal stem cells, the endothelial progenitor cells and the endothelial cells are derived from the same primary culture in one same well, from a single sample.

In one embodiment of the invention, the mesenchymal stem cells, the endothelial progenitor cells, and the endothelial cells are derived from a single sample from an individual.

In one embodiment of the invention, all the cells are derived from one same sample from a single subject.

In one embodiment of the invention, the cells are derived from a healthy individual i.e. not suffering from a disease, in particular a disease affecting the integrity of the bone marrow and/or related to a hematopoiesis disorder.

In another embodiment of the invention, the cells used are derived from an individual suffering from a disease, in particular a disease affecting the integrity of the bone marrow and/or related to a hematopoiesis disorder such as bone marrow aplasia, myelodysplastic syndrome, primary immunodeficiency or a hemopathy.

In one embodiment of the method, step a) is preceded by a step a0) at which the mesenchymal stem cells (MSCs), the endothelial progenitor cells and endothelial cells are selected and amplified together in one same medium and preferably in one same culture container. This medium is preferably EGM2 medium (Endothelial Growth Medium 2). This step lasts between 3 and 30 days, preferably between 5 and 25 days, and more particularly between 10 and 20 days.

The temperature of the culture method is chosen to permit culture of the cells. Typically a temperature for the cell culture is between 30° C. and 38° C.

The oxygen concentration is chosen to permit culture of the cells. Typically the oxygen concentration is between 10 and 30%, preferably from 15 to 25%. Also, the carbon dioxide concentration is between 2 and 8%.

In one embodiment of the invention, the cells are cultured in 2 dimensions or in 3 dimensions. The inventors have notably developed two separate 3-dimensional culture approaches. In the first, the different cell types form spheroids or organoids via self-organization, in the second the cells are deposited on a 3D substrate.

Therefore, in one embodiment of the method of the invention, the cells are seeded at step a) on a substrate in 3 dimensions, preferably in the form of spheroids or on a 3-dimensional substrate. The substrate can therefore be any substrate allowing the culture of the cells under consideration. In particular, the substrate can be a gel such as a hydrogel, or a solid. It can be formed from a silicone polymer (such as polydimethylsiloxane PDMS), a resin (such as DS 3000) and/or a calcium-based biomaterial. In one particular embodiment, the substrate is integrated in a microfluidic chip. Preferably, the substrate is a calcium-based biomaterial, preferably based on Tricalcium phosphate and/or hydroxyapatite, and more particularly the calcium-based biomaterial is formed from β-TCP.

In one embodiment of the invention, the differentiation of the mesenchymal stem cells into osteoblasts and adipocytes, as well as the organization of a vascular network, are obtained simultaneously on the biomaterial or within the organoid, preferably in a single step, in a single culture container, from a single sample, without posterior assembling of cells.

With regard to culture on a 3-dimensional substrate, the seeding as well as the culture and differentiations can be carried out in a perfusion bioreactor. This embodiment ensures good cell homogeneity on the biomaterial and allows the culture to be maintained for at least 3 weeks by means of the supply of oxygen and nutrients provided by the perfusion. This is particularly the case for cultures on microfluidic chips which are perfusion microbioreactors.

The inventors have observed that in the medium of the invention, the mesenchymal stem cells differentiate into osteoblasts and adipocytes, and the endothelial progenitor cells and endothelial cells form vessels, thereby reconstituting in vitro the microenvironment of bone marrow.

The inventors have observed that in the medium of the invention, the mesenchymal stem cells differentiate into osteoblasts and adipocytes and that a network of endothelial cells is organized, thereby reconstituting in vitro the microenvironment of bone marrow.

Method for Producing a Bone Marrow Reconstitution

The invention therefore concerns a method for producing a bone marrow reconstitution comprising the in vitro culture method of mesenchymal stem cells described above.

The invention concerns a method for producing a bone marrow reconstitution comprising the in vitro culture method of mesenchymal stem cells and/or a mixture of mesenchymal stem cells, endothelial progenitor cells and endothelial cells described above.

In one embodiment, these methods are preceded by an expansion step of the cells possibly lasting from 1 to 2 weeks.

Preferably, the cells are cultured in the culture medium of the invention for 4 to 20 days, more preferably from 7 to 15 days.

The temperature of the culture methods is chosen to allow culture of the cells. Typically, a temperature for the cell culture is between 30° C. and 38° C.

The oxygen concentration is chosen to allow culture of the cells. Typically, the oxygen concentration is between 10 and 30%, preferably of 15 to 25%. Similarly, the carbon dioxide concentration is between 2 and 8%.

In one preferred embodiment of the invention, the method for producing a bone marrow reconstitution comprises an in vitro culture method of mesenchymal stem cells, comprising the steps of:
a) seeding the mesenchymal stem cells in a culture medium of the invention with endothelial progenitor cells and endothelial cells; and
b) culturing said cells.

In this embodiment, the cells are cultured at step b) for 4 to 20 days, more preferably for 7 to 15 days.

In one preferred embodiment of the invention, the method for producing a bone marrow reconstitution comprises an in vitro culture method of mesenchymal stem cells and/or a mixture of mesenchymal stem cells, endothelial progenitor cells and endothelial cells, comprising the steps of:
a) seeding the mesenchymal stem cells and/or the mixture of mesenchymal stem cells, endothelial progenitor cells and endothelial cells in a culture medium of the invention; and
b) culturing said cells.

In this embodiment, the cells are cultured at step b) for 4 to 20 days, more preferably for 7 to 15 days.

The inventors, at protein level, have evidenced the presence in their bone marrow reconstitution of the three medullary compartments.

Bone Marrow Reconstitution

The invention concerns a bone marrow reconstitution able to be obtained with the production methods of the invention.

The invention also concerns a bone marrow reconstitution comprising osteoblasts, adipocytes, and vessel-forming endothelial cells.

The invention also concerns a bone marrow reconstitution comprising osteoblasts, adipocytes, and a network of endothelial cells. Preferably, the cells of the bone marrow reconstitution are in direct contact. In particular, the bone marrow reconstitution of the invention comprises vessels.

The reconstitution of bone marrow can therefore be in two or three dimensions and may optionally comprise the substrate or biomaterial used for formation thereof. Preferably, the reconstitution is included in a pharmaceutically acceptable medium.

The bone marrow reconstitution of the invention is typically adapted for implantation in the body of an individual.

Composition

The invention also concerns a composition comprising the cells obtained with the culture method of the invention. The composition of the invention is preferably liquid, and more preferably injectable. In this composition, the cells can be dispersed in a suspension, or in the form of spheroids. In this latter case, the spheroids have a mean diameter of less than 500 μm.

By «spheroids», it is meant a grouping of cells aggregated in the three dimensions in space. Preferably, a spheroid comprises from 500 to 750 000 cells, or even 1 000 to 500 000 cellules. The spheroids of the composition of the invention have a mean diameter of between 50 μm and 750 μm, preferably between 100 μm and 500 μm.

In one embodiment, the composition is a pharmaceutical composition which comprises at least one cell obtained with the culture method of the invention in a pharmaceutically acceptable medium.

By “pharmaceutically acceptable”, it is meant herein compositions and molecular entities which do not produce secondary, allergic, or other non-desirable reactions when administered to a subject. A pharmaceutically acceptable excipient or vehicle is therefore an encapsulating material, a diluent, a substrate, or any other non-toxic liquid, semi-solid or solid formulation auxiliary.

The compositions of the invention are typically prepared so that they adapt to the administration mode. The pharmaceutically acceptable excipients are typically partly determined by the administered composition, and by the particular technique used to a administer the composition.

The compositions of the invention are preferably liquid and adapted to the route of administration.

The pharmaceutical composition of the invention may also comprise at least one other active compound, for example a calcium-based biomaterial. Calcium-based biomaterials are known to be osteoinductive. They are already used in particular as fillers for loss of bone substance.

Use of the Bone Marrow Reconstitution or of the Composition

The subject of the invention is the bone marrow reconstitution of the invention or the composition of the invention for use thereof in the treatment of diseases.

Preferably, the diseases are diseases affecting the integrity of the bone marrow and/or related to a hematopoiesis disorder.

The bone marrow reconstitution or the composition of the invention could be used to promote hematopoiesis in various pathological situations, and in particular to allow ectopic hematopoiesis which would allow hematopoietic «normalization» in these patients.

By «treatment» or «to treat», it is meant herein partially or substantially to achieve one or more of the following results: partial or full reduction of the disease, improvement in a clinical symptom or indicator associated with the disease, the delaying, inhibiting or preventing of progression of the disease, or the partial or full delaying, inhibiting or preventing of onset of relapse of the disease.

A method for treating a disease affecting the integrity of the bone marrow and/or related to a hematopoiesis disorder is therefore proposed, wherein a therapeutically effective amount of bone marrow reconstitution of the invention or of the composition of the invention is administered to a subject suffering from a disease affecting the integrity of the bone marrow and/or related to a hematopoiesis disorder.

By «subject», it is meant herein a mammal, preferably a primate and more preferably a human. Preferably, the treated subject in the invention suffers from a disease affecting the integrity of the bone marrow and/or a hematopoiesis disorder.

By «disease affecting the integrity of the bone marrow and/or a hematopoiesis disorder», it is meant diseases in which the bone marrow is damaged and/or diseases related to an excess, deficiency, or disruption of hematopoiesis in an individual. For example, these diseases include bone marrow aplasia, myelodysplastic syndrome, primary immunodeficiency, and hemopathy such as leukemia, a lymphoma or myeloma. By «therapeutically effective amount», it is meant herein a sufficient amount of composition or reconstitution to disrupt, modify, control, or eliminate the disease. A «therapeutically effective amount» also designates an amount allowing the extent of the disease to be delayed or minimized. It also refers to the amount providing a therapeutic benefit in the treatment or management of the disease. Finally, the expression «therapeutically effective amount» means an amount of the composition or reconstitution, alone or in combination with other therapies, which provides a therapeutic benefit in the treatment or management of the disease, including an improvement in the symptoms associated with the disease.

The therapeutically effective amount is naturally dependent on the product administered, the mode of administration, the therapeutic indication, patient age, and patient condition.

The determination of administration route and dosage adapted to the subject lies within the reach of the persons skilled in the art.

The dosage is dependent on individual cases and, as is well known by persons skilled in the art, it must be adapted to individual circumstances to obtain a therapeutically effective amount and optimum effect. The level of a therapeutically effective amount is specific to each patient and will depend in particular on a variety of factors including the disorder being treated and severity of the disorder, patient age, weight, general state of health, gender and food diet, time of administration, administration route, duration of treatment, medication used in combination, and similar factors well known in the medical field.

Preferably, the bone marrow reconstitution or composition of the invention are administered via subcutaneous or intrafemoral route.

In another aspect, the subject of the invention is the use of a bone marrow reconstitution such as defined above in a biomedical application, wherein the biomedical application is preferably selected from among prostheses and medical devices. One subject of the invention is therefore the use of a bone marrow reconstitution such as defined above in a prosthesis or medical device.

In another aspect, the invention concerns the use of a bone marrow reconstitution such as previously defined as model for physiology and physiopathology studies, for the testing of compounds and/or physical and/or mechanical conditions.

By «physiology study» it is meant the study of mechanisms involved in cell interactions, the functioning and development of bone marrow throughout life.

The mechanisms involved in cell interactions within the bone marrow still remain ill-known, whether in physiology or pathology. The understanding of these mechanisms is limited due to the absence of an in vitro tool allowing the study of human bone marrow within a global context particularly integrating the microenvironmental aspects thereof. In particular, the use of primary cell cultures in the reconstitution of the invention allows closer insight into in vivo conditions.

The bone marrow reconstitution of the invention can be used to study the role of the constituent cellular and humoral elements thereof.

Therefore, a further aspect of the invention concerns the use of a bone marrow reconstitution of the invention to study the cellular and/or molecular mechanisms involved in the differentiation of mesenchymal stem cells.

Since the reconstitution can be formed from a donor sample, it can be used to study variations due to age, gender, etc. . . . It can also serve as bone marrow model for a particular stage in life, for example an elderly bone marrow model, young bone marrow model, fetal bone marrow model, etc.

By «physiopathology study» it is meant studying the impact of diseases on the characteristics of bone marrow, such as cell morphology, cell growth, the formation or disappearance of vessels, the expression of some proteins, etc. . . . . In this embodiment, the reconstitution then comprises at least one model cell type of the studied disease and/or a cell type derived from an individual suffering from the disease being studied.

A further subject of the invention is therefore the use of a bone marrow reconstitution of the invention as pathological bone marrow model. In one particular embodiment, one or more cell types of the reconstitution are pathological model cell types. By «pathological model cell types», it is meant cell types derived from animal models reproducing pathologies occurring spontaneously or induced by genetic engineering methods (e.g. transgenesis) or with pharmacological tools, to reproduce the characteristics of cells of individuals suffering from these particular pathologies.

In one particular embodiment, one or more cell types of the reconstitution are derived from an individual suffering from the disease being studied.

Preferably, the studied pathologies of the invention are pathologies having or suspected of having an impact on the bone marrow, such as: bone marrow aplasia, myelodysplastic syndrome, primary immunodeficiency, hemopathies such as leukemia, lymphoma, or myeloma.

The reconstitution of the invention can also be used to study pathologies which develop at a particular moment in the life of an individual.

By «testing molecules» it is meant studying the impact of these molecules on the bone marrow. In this embodiment, at least one molecule to be tested is applied to the reconstitution of the invention and, after an exposure or incubation time, the reconstitution is analyzed to determine changes caused by said at least one tested molecule. In particular, these changes can concern cell morphology, cell growth and death, the formation or disappearance of vessels, the expression of certain proteins, cell differentiation, etc. . . . .

By «molecules» it is meant preventive, therapeutic or diagnostic molecules targeting cellular and molecular players of the bone marrow.

Therefore, in a further aspect, the invention concerns the use of a bone marrow reconstitution of the invention to study the efficacy and/or toxicity of a candidate medication.

In one embodiment, the invention concerns the use of a bone marrow reconstitution of the invention to study the efficacy and/or toxicity of a candidate medication for a particular individual. A bone marrow reconstitution formed from a sample of the individual can be used as model to study the efficacy and/or toxicity of a candidate medication for this individual in particular. This type of analysis can particularly be carried out as part of personalized medicine, to define the best adapted treatment for an individual.

By «testing physical and/or mechanical conditions», it is meant to study the impact of these conditions on the bone marrow reconstitution. In this embodiment, at least one physical or mechanical condition is applied to the reconstitution of the invention. After an exposure or incubation time, the reconstitution is analyzed to determine the changes caused by said at least one tested physical or mechanical condition. These changes can particularly concern cell morphology, cell growth and death, the formation or disappearance of vessels, the expression of certain proteins, cell differentiation, etc. . . . The effect of the added physical or mechanical condition can particularly be studied by comparison with a reconstitution to which the condition was not applied.

By «physical condition», it is particularly meant the use of waves such as magnetic, electromagnetic waves or ultrasound.

By «mechanical condition» it is particularly meant pressure, contraction, stretching, gravity, weightlessness, shear.

The invention concerns the use of a bone marrow reconstitution of the invention as model for the long-term study of hematopoiesis and/or differentiation of mesenchymal stem cells.

By «long-term», it is meant over more than 3 days, more than 10 days, more than 15 days, more than 20 days and preferably up to 21 days.

By «hematopoiesis study», it is meant study of the proliferation and differentiation of blood cells.

In the present application, the term «comprising» is to be interpreted as covering all the characteristics specifically mentioned and optionally some additional non-specified characteristics. Additionally, use of the term «comprising» also describes the embodiment in which there is no characteristic other than those specifically mentioned (i.e. «consisting of»).

The present invention is illustrated in more detail in the Figures and examples below.

### FIGURES

FIG. 1 gives the relative expression of genes involved in osteoblastic, adipocyte and vascular lineages (endothelial cells) contained in the bone marrow reconstitutions after culture in 2D models. After selection and amplification in EGM2 medium, the cells were cultured, without passaging, in one same culture container for 14 days in 2D in the medium of the invention (with FCS and intralipids). Gene expression was quantified by Reverse Transcriptase-quantitative Polymerase Chain Reaction (RT-qPCR) and calculated after normalization with a reference gene using the method 2^{-\hat{ }CT}, n=5.

FIG. 2 gives the relative expression of genes involved in osteoblastic, adipocyte and vascular lineages (endothelial cells) contained in the bone marrow reconstitutions after culture in 2D models. After selection and amplification in EGM2 medium, the cells were cultured, without passaging, in one some culture container for 14 days in 2D, either in a medium with FCS and intralipids, or in a PL medium without intralipids, or in their amplification medium (EGM2 medium) corresponding to the CTRL condition. Gene expression was quantified by Reverse Transcriptase-quantitative Polymerase Chain Reaction (RT-qPCR) and calculated after normalization with a reference gene using the method 2^{-\hat{ }CT}, n=3.

FIG. 3 gives the relative expression of genes corresponding to the pro-hematopoietic factors present in the bone marrow reconstitutions after culture in 2D models. After selection and amplification in EGM2 medium, were cultured without passaging in one same culture container for 14 days in 2D, either in a medium with FCS and intralipids, or in a PL medium without intralipids, or in their amplification medium (EGM2 medium) corresponding to the CTRL condition. Gene expression was quantified by Reverse Transcriptase-quantitative Polymerase Chain Reaction (RT-qPCR) and calculated after normalization with a reference gene using the method 2^{-\hat{ }CT}, n=3.

FIG. 4 gives the number of CD34⁺/CD38⁻ cells, the most immature hematopoietic cells obtained after placing hematopoietic stem cells (HSCs) in co-culture for 14 days with the bone marrow reconstitutions obtained according to the method described in the invention.

### EXAMPLES

### Example 1

The mesenchymal stem cells (MSCs), endothelial progenitor cells and endothelial cells derived from one same bone marrow sample were selected and amplified by means of the adherent properties of these cell types and use of the Endothelial Growth Medium 2 (Promocell) for culture.

After 14-day culture, these cells were lifted and used to generate different bone marrow models in 2 and 3 dimensions. For this purpose, the cells were cultured in a medium allowing differentiation in the same culture of one portion of the MSCs into osteoblasts (bone compartment), and of another portion into adipocytes (adipose compartment), whilst allowing the organization of a vascular network. The medium used here was composed of MEMα base medium supplemented with 2% fetal calf serum, 50 uM ascorbic acid, 0.04% (v/v) intralipids, 50 ng/mL Bone Morphogenetic Protein, 7, 5 μg/mL insulin, 10 μg/mL apotransferrin and 10 ng/mL VEGF.

For the 3D models, two technologies were used: the generation of spheroids based on cell self-organization, and the use of a cells/biomaterials complex placed under perfusion in a bioreactor, where the biomaterial was βTCP.

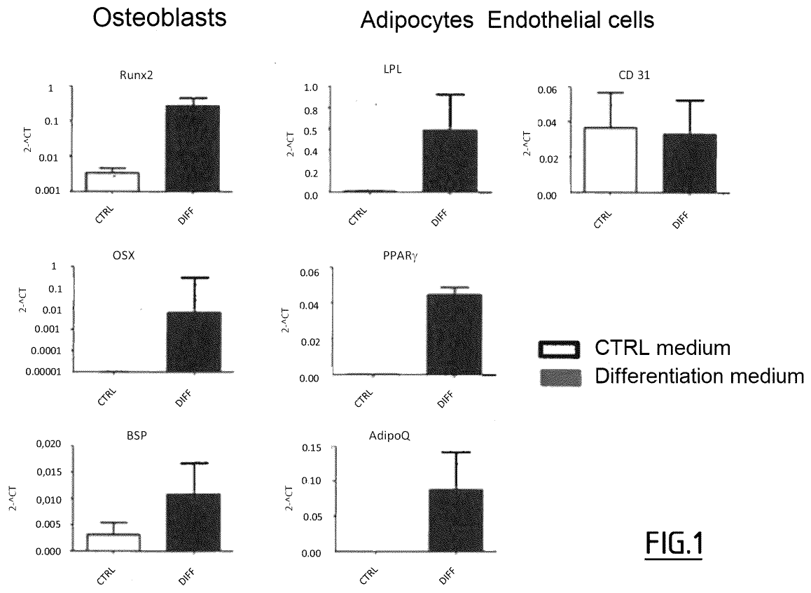

The inventors evidenced the presence of the different compartments by gene analyses (RT-qPCR). FIG. 1 gives these results on the 2D model.

The inventors also evidenced the presence of the different compartments by fluorescence analyses (immunofluorescence and fluorescent probes), by targeting specific markers of each compartment (Osterix for osteoblasts, CD31 for endothelial cells, BODIPY for adipocytes). These different markers were evidenced in the 2D model, in the spheroid model (3D model) and in the βTCP biomaterial model in a perfusion bioreactor (3D model).

### Example 2

The bone marrow reconstitutions were prepared as in Example 1, but different compositions of the differentiation medium were tested.

The cells recovered after a primary culture of bone marrow in EGM2 were seeded at 20 000 cells/cm 2 in EGM2 medium and left in this medium for 4 to 6 days, at 37° C. and 5% CO₂. The EGM2 medium was then replaced by one of the two differentiation media (FCS or PL) described below. The differentiation medium was renewed at the rate of 2 changes per week. After 14 days in the differentiation medium, culturing was halted to carry out gene analysis.

The so-called FCS medium was composed of a base MEMα medium supplemented with 2% (v/v) fetal calf serum, 50 μM ascorbic acid, 0.04% (v/v) intralipids, 50 ng/mL Bone Morphogenetic Protein, 7, 5 μg/mL insulin, 10 μg/mL apotransferrin and 10 ng/mL VEGF.

The so-called PL medium was composed of MEMα base medium supplemented with 1% (v/v) platelet lysate (PL), 50 μM ascorbic acid, 50 ng/mL Bone Morphogenetic Protein 7, 5 μg/mL insulin, 10 μg/mL apotransferrin and 10 ng/mL VEGF.

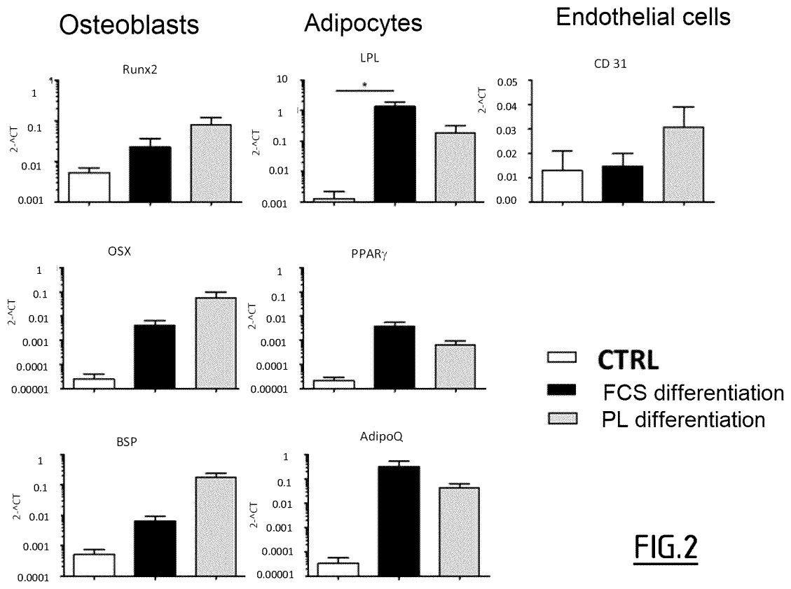

The simultaneous generation of the 3 medullary compartments was evaluated in a 2D culture by gene analysis (see FIG. 2).

The results show that there is no significant difference between the two tested differentiation media. Each of the two media allows in vitro differentiation of one portion of the MSCs into osteoblasts, and of another portion into adipocytes, whilst allowing maintaining of the positive CD31 cells corresponding to the endothelial compartment (FIG. 2).

In addition, the inventors observed a trend. It would seem that the FCS medium differentiates more MSCs into adipocytes, and the LP medium differentiates more MSCs into osteoblasts. These results suggest an adaptability/flexibility of the method. Several studies have effectively shown that the differentiation of MSCs is modified by physical and mechanical stresses, which is to be taken into consideration for the 3D models in which these stresses differ from those of standard 2D models (varying rigidity of biomaterials, varying contraction of spheroids/organoids, etc. . . . ). The production method could therefore allow adaptation to these stresses by adapting the differentiation medium.

Also, the older the bone marrow the more the adipose tissue increases in proportion and loses functionality with a drop in vascularization; conversely the younger the bone marrow the greater the bone density with stronger vascularization. Therefore, modulation of the composition of the culture medium could allow studies on ageing at the hematopoietic niche.

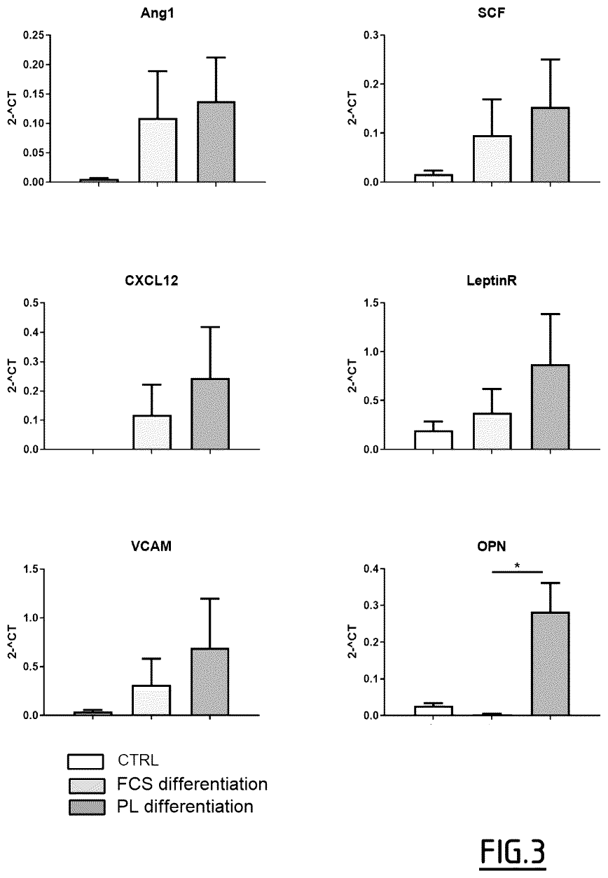

Further, the results show that with the method of the invention there is an increase in the expression of the main pro-hematopoietic factors in the bone marrow reconstitutions generated in vitro, compared with the CTRL condition (FIG. 3). These factors are secreted or expressed in vivo by the medullary microenvironment at the hematopoietic niches, and are needed for the setting up of hematopoiesis in the bone marrow, indicating the functionality of the bone marrow reconstitutions of the invention.

### Example 3

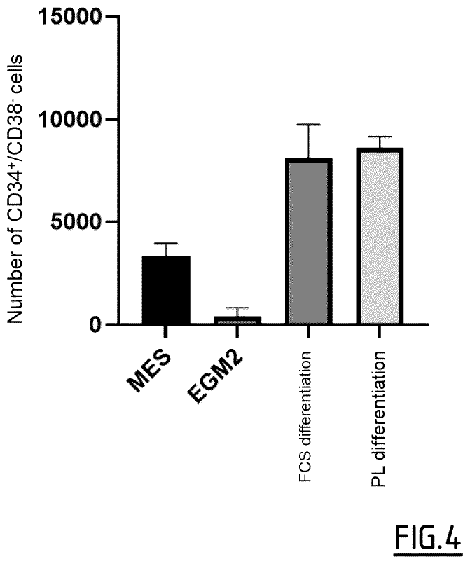

To reinforce the functional study on in vitro bone marrow reconstitutions, the inventors tested the addition of hematopoietic stem cells to these bone marrow reconstitutions, to evaluate hematopoiesis in vitro (FIG. 4).

Formation of bone marrow reconstitutions: After selection and amplification in EGM2 medium, the mesenchymal stem cells and/or the mixture of mesenchymal stem cells, endothelial progenitor cells and endothelial cells were cultured, without passaging, in one same culture container for 14 days in 2D, either in a medium with FCS and intralipids, or in a medium with PL without intralipids, or in their amplification medium (EGM2 medium) corresponding to the condition «EGM2». In parallel, after pre-selection and pre-amplification in MEMα medium with FCS, the mesenchymal stem cells and/or the mixture of mesenchymal stem cells, endothelial progenitor cells and endothelial cells were cultured under the same conditions as previously (same cell density, culture time, etc.) for 14 days in 2D in MEMα medium with FCS. Since this medium is a reference medium used in most publications for the culture of mesenchymal stem cells, it corresponds to the condition «MES».

Co-culture with hematopoietic stem cells (HSCs): The HSCs were sorted from a unit of placenta blood on the base of the positive CD34 marker. They were then placed in co-culture with the previously generated 2D bone marrow reconstitutions, for 14 days in a medium of IMDM+10% FCS+1 μM hydrocortisone. On completion of the co-culture, all the cells were collected, counted, and analyzed by flow cytometry (n=3).

The first results show better maintaining and proliferation of the most immature hematopoietic cells (CD34⁺/CD38⁻) with the bone marrow reconstitutions of the invention than in the control conditions. In numerous models, most of these cells differentiate rapidly and are scarcely maintained.

1) A culture medium allowing the obtaining in a single step, in the same culture container, of cells differentiated into osteoblasts and adipocytes, as well as a network of organized endothelial cells from the same pool of mesenchymal stem cells and/or a mixture of mesenchymal stem cells, endothelial progenitor cells and endothelial cells, comprising:
a) fetal calf serum (FCS) and/or platelet lysate (PL);
b) 10-100 μM ascorbic acid;
c) 10-100 ng/mL «Bone Morphogenetic Protein 7 » (BMP7);
d) 1-10 μg/mL insulin;
e) 1-50 μg/mL apotransferrin; and
f) 1-100 ng/mL vascular endothelial growth factor (VEGF); and optionally
g) 0.01-0.5% (v/v) intralipids.

2) An in vitro culture method of mesenchymal stem cells and/or a mixture of mesenchymal stem cells, endothelial progenitor cells and endothelial cells, comprising the steps of:
a) seeding the mesenchymal stem cells and/or the mixture of mesenchymal stem cells, endothelial progenitor cells and endothelial cells in a culture medium such as defined in claim 1; and
b) culturing said mesenchymal stem cells and/or said mixture of mesenchymal stem cells, endothelial progenitor cells and endothelial cells.

3) The culture method according to claim 2, wherein one portion of the mesenchymal stem cells is differentiated into osteoblasts and another portion of the mesenchymal stem cells is differentiated into adipocytes simultaneously, in a single step, in the same medium and in the same culture container, without posterior assembling of cells.

4) The culture method according to claim 2, wherein endothelial progenitor cells and endothelial cells organize themselves into vessels in the culture medium, at the same time as said mesenchymal stem cells differentiate.

5) The culture method according to claim 2, wherein the cells are primary cells.

6) The culture method according to claim 2, wherein the cells are all derived from one same sample from a single subject.

7) The culture method according to claim 2, wherein at step a) the cells are seeded in 3 dimensions in the form of spheroids or on a 3-dimensional substrate.

8) A method for producing a bone marrow reconstitution comprising the in vitro culture method of mesenchymal stem cells and/or a mixture of mesenchymal stem cells, endothelial progenitor cells and endothelial cells according to claim 2, wherein the cells are cultured at step b) for 4 to 20 days.

9) A bone marrow reconstitution obtained with the method in claim 8.

10) A bone marrow reconstitution comprising osteoblasts, adipocytes, and vessel-forming endothelial cells.

11) A composition comprising cells obtained with the method of claim 2.

12) A method for treating a disease affecting the integrity of the bone marrow and/or related to a hematopoiesis disorder, wherein a therapeutically effective amount of a bone marrow reconstitution according to claim 9 is administered to a subject suffering from a disease affecting the integrity of the bone marrow and/or related to a hematopoiesis disorder.

13) A model for physiology, or physiopathology studies comprising the bone marrow reconstitution of claim 9.

14) A prosthesis or medical device comprising the bone marrow reconstitution such as defined in claim 9.

15) A method for treating a disease affecting the integrity of the bone marrow and/or related to a hematopoiesis disorder, wherein a therapeutically effective amount of a bone marrow reconstitution according to claim 10 is administered to a subject suffering from a disease affecting the integrity of the bone marrow and/or related to a hematopoiesis disorder.

16) A Method for treating a disease affecting the integrity of the bone marrow and/or related to a hematopoiesis disorder, wherein a therapeutically effective amount of a composition according to claim 11 is administered to a subject suffering from a disease affecting the integrity of the bone marrow and/or related to a hematopoiesis disorder.
